(19) United States
(12) Patent Application Publication
(10) Pub. No.: US 2023/0356225 A1
(43) Pub. Date: Nov. 9, 2023

(54) MICROFLUIDIC DEVICES FOR PARTICLE CAPTURE AND METHODS THEREOF

(71) Applicant: Georgia Tech Research Corporation, Atlanta, GA (US)

(72) Inventors: Emily L. Jackson-Holmes, Atlanta, GA (US); Gongchen Sun, Atlanta, GA (US); Guillaume A. Aubry, Atlanta, GA (US); Hang Lu, Atlanta, GA (US)

(21) Appl. No.: 18/246,243

(22) PCT Filed: Sep. 27, 2021
(86) PCT No.: PCT/US2021/052181

§ 371 Date: Mar. 22, 2023

Related U.S. Application Data
(60) Provisional application No. 63/084,245, filed on Sep. 28, 2020.

(51) Int. Cl. B01L 3/00

(52) U.S. Cl. CPC B01L 3/502761; B01L 2200/0652; B01L 2300/0883; B01L 2300/087; B01L 2400/0487; B01L 2400/086

(57) ABSTRACT

Microfluidic devices for capturing particles, such as cells, are disclosed. An example device includes a main fluid channel, a first interaction chamber in fluid communication with the main fluid channel, a first particle trap extending from a first wall of the main fluid channel and positioned between the main fluid channel and the first interaction chamber, and a second particle trap extending from a second wall of the main fluid channel and positioned between the main fluid channel and the first interaction chamber. The first particle trap and the second particle trap are in fluid communication both via the first interaction chamber and via the main fluid channel. Methods of using the devices are also disclosed.

### CROSS-REFERENCE TO RELATED APPLICATIONS

This application claims priority to U.S. Provisional Patent Application No. 63/084,245, filed 28 Sep. 2020, which is hereby incorporated by reference herein in its entirety as if fully set forth below.

### STATEMENT OF RIGHTS UNDER FEDERALLY SPONSORED RESEARCH

This invention was made with government support under. Grant No. EEC-1648035 awarded by the National Science Foundation. The government has certain rights in the invention.

### FIELD OF THE DISCLOSURE

Embodiments of the present disclosure relate generally to microfluidic devices and methods of using microfluidic devices and, more particularly, to microfluidic devices and methods of using microfluidic devices for capturing particles, such as cells, for observation.

### BACKGROUND

The ability to view and/or analyze the interaction of two or more particles, such as cells, is an important process for developing many treatments. For example, analyzing the interaction of CAR-T cells with cancer cells is an important step in developing immunotherapy regimes. The ability to capture and combine these cells into a defined space, however, presents difficulties.

Prior methods for capturing and combining cells, for example, include manual selection and combination of cells, though these methods can be tedious. Other methods for capturing and combining cells using microfluidic technologies involved many components, such as valves to direct fluid flow. None of the prior device-assisted methods were able to pair different number of two or more cell types in a passive manner, i.e., enabled by the flow of fluid through the device alone. These and other drawbacks exist.

### SUMMARY

Briefly described, embodiments of the present disclosure relate generally to microfluidic devices and methods of using microfluidic devices. More particularly, the present disclosure provides microfluidic devices and methods of using microfluidic devices for capturing particles, such as cells, for observation.

A first embodiment of the present disclosure provides a device. The device can include a main fluid channel. The device can include a first interaction chamber in fluid communication with the main fluid channel. The device can include a first particle trap extending from a first wall of the main fluid channel and positioned between the main fluid channel and the first interaction chamber. The device can include a second particle trap extending from a second wall of the main fluid channel and positioned between the main fluid channel and the first interaction chamber. The first particle trap and the second particle trap can be in fluid communication both via the first interaction chamber and via the main fluid channel.

In any of the embodiments described herein, the main fluid channel can have a serpentine shape.

In any of the embodiments described herein, the device can include a first restriction channel positioned between the first particle trap and the first interaction chamber. The first restriction channel can have a first channel cross-section smaller than a second channel cross-section of the first particle trap.

In any of the embodiments described herein, the device can include a second restriction channel positioned between the second particle trap and the first interaction chamber. The second restriction channel can have a third channel cross-section smaller than a fourth channel cross-section of the second particle trap.

In any of the embodiments described herein, at least a portion of the main fluid channel, the first interaction chamber, the first particle trap, the second particle trap, the first restriction channel, and the second restriction channel can be coplanar.

In any of the embodiments described herein, the device can include a third particle trap extending from the first wall of the main fluid channel and positioned between the main fluid channel and the first interaction chamber.

In any of the embodiments described herein, the first particle trap and the third particle trap can be parallel and coplanar.

In any of the embodiments described herein, the device can include a first restriction channel positioned between the first particle trap and the first interaction chamber. The first restriction channel can have a first channel cross-section smaller than a second channel cross-section of the first particle trap. The device can include a second restriction channel positioned between the third particle trap and the first interaction chamber. The second restriction channel can have a third channel cross-section smaller than a fourth channel cross-section of the third particle trap.

In any of the embodiments described herein, the first particle trap can be one of a plurality of particle traps extending from the first wall of the main fluid channel and positioned between the main fluid channel and the first interaction chamber.

In any of the embodiments described herein, the first particle trap can be one of a plurality of particle traps extending from the second wall of the main fluid channel and positioned between the main fluid channel and the first interaction chamber.

In any of the embodiments described herein, at least a portion of the main fluid channel, the first interaction chamber, the first particle trap, and the second particle trap can be coplanar.

In any of the embodiments described herein, the device can include a second interaction chamber in fluid communication with the main fluid channel. The device can include a third particle trap extending from the first wall of the main fluid channel and positioned between the main fluid channel and the second interaction chamber. The third particle trap can be positioned linearly from the first particle trap along the first wall. The device can include a fourth particle trap extending from the second wall of the main fluid channel and positioned between the main fluid channel and the second interaction chamber. The fourth particle trap can be positioned linearly from the second particle trap along the second wall.

In any of the embodiments described herein, at least a portion of the main fluid channel, the first interaction chamber, the first particle trap, the second particle trap, the third particle trap, and the fourth particle trap can be coplanar.

In any of the embodiments described herein, the first interaction chamber can be one of a plurality of interaction chambers, wherein the plurality of interaction chambers comprise at least some interaction chambers having different quantities of particle traps.

In any of the embodiments described herein, the first particle trap can be configured to receive a first particle from the main fluid channel and pass the first particle to the first interaction chamber when fluid is flowing through the main fluid channel in a first direction. The second particle trap can be configured to receive a second particle from the main fluid channel and pass the second particle to the first interaction chamber when fluid is flowing through the main fluid channel in a second direction.

In any of the embodiments described herein, the first particle trap can be configured to receive a first particle from the main fluid channel and pass the first particle to the first interaction chamber when fluid is flowing through the main fluid channel in a first direction. The second particle trap is blocked by the first particle when the fluid is flowing in the first direction.

In any of the embodiments described herein, the device can include a chamber ramp positioned within the first interaction chamber proximate the second particle trap and configured to retain a particle within the first interaction chamber.

Another embodiment of the present disclosure provides a method for loading cells into the devices described herein. The method can include delivering a first fluid comprising a first cell to the main fluid channel. The method can include routing the first fluid in a first direction through the main fluid channel such that the first cell is captured in the first particle trap and passes to the first interaction chamber. The method can include delivering a second fluid comprising a second cell to the main fluid channel. The method can include routing the second fluid in a second direction through the main fluid channel such that the second cell is captured in the second particle trap and passes to the first interaction chamber.

Another embodiment of the present disclosure provides a method for manufacturing the device(s) described herein. The method can include molding the main fluid channel, the first particle trap, the second particle trap, and the first interaction chamber into a planar surface of a substrate.

Another embodiment of the present disclosure provides a method for manufacturing the device(s) described herein. The method can include etching the main fluid channel, the first particle trap, the second particle trap, and the first interaction chamber into a planar surface of a substrate.

Another embodiment of the present disclosure provides a device. The device can include a main fluid channel having a serpentine shape. The main fluid channel can include a first opening and a second opening, wherein the first opening acts both as a first fluid inlet and a first fluid outlet. The second opening can act both as a second fluid inlet and a second fluid outlet. The device can include a first interaction chamber in fluid communication with the main fluid channel. The device can include a first particle trap extending from a first wall of the main fluid channel and positioned between the main fluid channel and the first interaction chamber. The device can include a first restriction channel positioned between the first particle trap and the first interaction chamber. The device can include a second particle trap extending from a second wall of the main fluid channel and positioned between the main fluid channel and the first interaction chamber. The device can include a second restriction channel positioned between the second particle trap and the first interaction chamber. The first particle trap and the second particle trap can be in fluid communication both via the first interaction chamber and via the main fluid channel.

In any of the embodiments described herein, at least a portion of the main fluid channel, the first interaction chamber, the first particle trap, the second particle trap, the first restriction channel, and the second restriction channel can be coplanar.

In any of the embodiments described herein, the first restriction channel can have a first channel cross-section smaller than a second channel cross-section of the first particle trap. The second restriction channel can have a third channel cross-section smaller than a fourth channel cross-section of the second particle trap.

In any of the embodiments described herein, the first particle trap can be configured to receive a first particle from the main fluid channel and pass the first particle to the first interaction chamber through the first restriction channel when fluid is flowing through the main fluid channel in a first direction. The second particle trap can be configured to receive a second particle from the main fluid channel and pass the second particle to the first interaction chamber through the second restriction channel when fluid is flowing through the main fluid channel in a second direction.

In any of the embodiments described herein, the device can include a second interaction chamber in fluid communication with the main fluid channel. The device can include a third particle trap extending from the first wall of the main fluid channel and positioned between the main fluid channel and the second interaction chamber. The third particle trap can be positioned linearly from the first particle trap along the first wall. The device can include a third restriction channel positioned between the third particle trap and the second interaction chamber.

In any of the embodiments described herein, the device can include a fourth particle trap extending from the second wall of the main fluid channel and positioned between the main fluid channel and the second interaction chamber. The fourth particle trap can be positioned linearly from the second particle trap along the second wall. The device can include a fourth restriction channel positioned between the fourth particle trap and the second interaction chamber.

In any of the embodiments described herein, the device can include a fifth particle trap extending from the first wall of the main fluid channel and positioned between the main fluid channel and the second interaction chamber, the fifth particle trap being positioned linearly from the first particle trap and the third particle trap along the first wall. The device can include a fifth restriction channel positioned between the fifth particle trap and the second interaction chamber.

In any of the embodiments described herein, the device can include a third particle trap extending from the first wall of the main fluid channel and positioned between the main fluid channel and the first interaction chamber. The device can include a third restriction channel positioned between the third particle trap and the first interaction chamber. A first channel cross-section of the first particle trap can be larger than a second channel cross-section of the third particle trap.

In any of the embodiments described herein, the first particle trap can be configured to trap larger cells within a fluid delivered to the first opening than the third particle trap.

Another embodiment of the present disclosure provides a method for loading cells into the device(s) described herein. The method can include delivering a first fluid comprising a first cell to the first opening. The method can include routing the first fluid in a first direction through the main fluid channel such that the first cell is captured in the first particle trap. The method can include increasing a first pressure of the first fluid, causing the first cell to pass through the first restriction channel and into the first interaction chamber. The method can include delivering a second fluid comprising a second cell to the second opening. The method can include routing the second fluid in a second direction through the main fluid channel such that the second cell is captured in the second particle trap. The method can include increasing a second pressure of the second fluid, causing the second cell to pass through the second restriction channel and into the first interaction chamber.

In any of the embodiments described herein, the method can include placing a slide onto a planar surface covering the main fluid channel, the first particle trap, the first restriction channel, the second particle trap, the second restriction channel, and the first interaction chamber. The method can include orienting the device such that the slide is placed below the device. The method can include performing an analysis of the first cell and second cell through a substrate of the device comprising the first interaction chamber.

Another embodiment of the present disclosure provides a method for manufacturing the device(s) described herein. The method can include molding the main fluid channel, the first particle trap, the first restriction channel, the second particle trap, the second restriction channel, and the first interaction chamber into a planar surface of a substrate.

Another embodiment of the present disclosure provides a method for manufacturing the device(s) described herein. The method can include etching the main fluid channel, the first particle trap, the first restriction channel, the second particle trap, the second restriction channel, and the first interaction chamber into a planar surface of a substrate.

Another embodiment of the present disclosure provides a device. The device can include a main fluid channel having a serpentine shape. The device can include a plurality of interaction chambers disposed along a length of the main fluid channel. At least a first interaction chamber of the plurality of interaction chambers can include a first trap in fluid communication with the main fluid channel at one end and the first interaction chamber at another end. At least the first interaction chamber of the plurality of interaction chambers can include a first plurality of particle outlet traps in fluid communication with the first interaction chamber at one end and the main fluid channel at another end. The main fluid channel can provide a fluid flow path along the serpentine shape such that the first plurality of particle outlet traps is positioned along the serpentine shape of the main fluid channel downstream from the first trap along the fluid flow path.

In any of the embodiments described herein, least a portion of the main fluid channel, the plurality of interaction chambers, the first trap, and the first plurality of particle outlet traps are coplanar.

In any of the embodiments described herein, the plurality of interaction chambers can include a second interaction chamber. The second interaction chamber can include a second trap in fluid communication with the main fluid channel at one end and the second interaction chamber at another end. The second interaction chamber can include a second plurality of particle outlet traps in fluid communication with the second interaction chamber at one end and the main fluid channel at another end.

In any of the embodiments described herein, the second plurality of particle outlet traps can have fewer traps than the first plurality of particle outlet traps.

In any of the embodiments described herein, the first plurality of particle outlet traps can be configured to be blocked by a plurality of cells within the first interaction chamber, thereby stopping flow through the first trap.

Another embodiment of the present disclosure provides a method for loading cells into the device(s) described herein. The method can include delivering a fluid comprising a plurality of cells into an inlet of the main fluid channel. The method can include advancing the fluid past the first trap such that at least a portion of the plurality of cells enters the first interaction chamber via the firs trap. At least a portion of the plurality of cells can block the first plurality of particle outlet traps and prohibits further flow into the first interaction chamber.

Another embodiment of the present disclosure provides a method for loading cells into the device(s) described herein. The method can include delivering a first fluid comprising a first cell into a first opening of the device. The method can include advancing the first fluid along a main fluid channel in a first direction. The method can include capturing the first cell in a first particle trap along the main fluid channel. The method can include increasing a first pressure of the first fluid, causing the first cell to pass through a first restriction channel and into a first interaction chamber, wherein the first restriction channel can have a first channel cross-section smaller than a second channel cross-section of the first particle trap.

In any of the embodiments described herein, the method can include delivering a second fluid comprising a second cell into a second opening of the device. The method can include advancing the second fluid along a main fluid channel in a second direction. The method can include capturing the second cell in a second particle trap along the main fluid channel. The method can include increasing a second pressure of the second fluid, causing the second cell to pass through a second restriction channel and into the first interaction chamber, wherein the second restriction channel can have a third channel cross-section smaller than a fourth channel cross-section of the second particle trap.

In any of the embodiments described herein, the method can include placing a slide onto a planar surface covering the main fluid channel, the first particle trap, the first restriction channel, the second particle trap, the second restriction channel, and the first interaction chamber. The method can include orienting the device such that the slide is placed below the device. The method can include performing an analysis of the first cell and second cell through a substrate of the device comprising the first interaction chamber.

In any of the embodiments described herein, the method can include delivering a third fluid comprising a third cell into the first opening of the device. The method can include advancing the third fluid along the main fluid channel in the first direction. The method can include capturing the third cell in a third particle trap along the main fluid channel. The method can include increasing a third pressure of the third fluid, causing the third cell to pass through a third restriction channel and into the first interaction chamber. The third restriction channel can have a third channel cross-section smaller than a fourth channel cross-section of the third particle trap.

In any of the embodiments described herein, the fourth channel cross-section of the third particle trap is a different size than the second channel cross-section of the first particle trap.

In any of the embodiments described herein, the first fluid further can include a second cell. The method can further include capturing the second cell in a second particle trap along the main fluid channel. Increasing the first pressure can cause the second cell to pass through a second restriction channel and into the first interaction chamber. The second restriction channel can have a third channel cross-section smaller than a fourth channel cross-section of the second particle trap. The fourth channel cross-section of the second particle trap can be a different size than the second channel cross-section of the first particle trap.

In any of the embodiments described herein, at least a portion of the main fluid channel, the first interaction chamber, the first particle trap, and the first restriction channel can be coplanar.

Another embodiment of the present disclosure provides a method for loading cells into a device. The method can include delivering a first fluid comprising a plurality of cells into a first opening of the device. The method can include advancing the first fluid along a main fluid channel in a first direction. The method can include capturing at least a first portion of the plurality of cells into a first interaction chamber via a first particle trap, causing a first particle outlet trap in fluid communication with the first interaction chamber to be blocked by the at least a first portion of the plurality of cells and redirecting the first fluid past the first particle trap. The method can include capturing at least a second portion of the plurality of cells into a second interaction chamber via a second particle trap.

In any of the embodiments described herein, the first portion of the plurality of cells can further block a second particle outlet trap in fluid communication with the first interaction chamber.

In any of the embodiments described herein, the second interaction chamber can have a different quantity of particle outlet traps than the first interaction chamber, thereby requiring a different quantity of cells to block flow into the second interaction chamber.

These and other aspects of the present disclosure are described in the Detailed Description below and the accompanying figures. Other aspects and features of embodiments of the present disclosure will become apparent to those of ordinary skill in the art upon reviewing the following description of specific, example embodiments of the present disclosure in concert with the figures. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments of the disclosure discussed herein. In similar fashion, while example embodiments may be discussed below as device, system, or method embodiments, it is to be understood that such example embodiments can be implemented in various devices, systems, and methods of the present disclosure.

### BRIEF DESCRIPTION OF THE FIGURES

Reference will now be made to the accompanying figures and diagrams, which are not necessarily drawn to scale, and wherein:

FIG. 1A is a schematic of a device for capturing particles, according to an exemplary embodiment of the present disclosure. FIG. 1B depicts an interaction chamber of the device shown in FIG. 1A. FIG. 1C depicts another interaction chamber of the device shown in FIG. 1A;

FIGS. 2A-2C are schematics of the layout of an example interaction chamber, according to an exemplary embodiment of the present disclosure. FIG. 2A is a top/bottom plan schematic of the interaction chamber and corresponding traps and channels. FIG. 2B is a cross sectional schematic of the interaction chamber. FIG. 2C is a perspective schematic of the interaction chamber;

FIGS. 3A-3C are schematics of example interaction chambers of the devices shown herein, according to an exemplary embodiment of the present disclosure. FIG. 3A shows an example device having a single trap on each side of the interaction chamber(s). FIG. 3B shows two traps on a first side of the interaction chamber(s). FIG. 3C shows three traps on a first side of the interaction chamber(s);

FIGS. 4A and 4B are cross sectional schematics showing example processes for analyzing cells within a device, according to an exemplary embodiment of the present disclosure;

FIGS. 5A-5D are schematics showing an example process for loading cells into an interaction chamber of a device, according to an exemplary embodiment of the present disclosure;

FIGS. 6A-6C are brightfield/fluorescent images showing cells being loaded into a device, according to an exemplary embodiment of the present disclosure. FIG. 6A shows K562 cells labeled with CellTracker Red loaded into outer cell traps. FIG. 6B shows Jurkat cells labeled with CellTracker Green loaded into outer cell traps. FIG. 6C shows three example interaction chambers into which both Jurkat and K562 cells have been loaded;

FIG. 7A is a schematic of a device for capturing particles, according to an exemplary embodiment of the present disclosure. FIG. 7B depicts an interaction chamber of FIG. 7A that includes five particle outlet traps. FIG. 7C depicts an interaction chamber of FIG. 7A that includes four particle outlet traps. FIG. 7D depicts an interaction chamber of FIG. 7A that includes three particle outlet traps. FIG. 7E depicts an interaction chamber of FIG. 7A that includes two particle outlet traps according to an exemplary embodiment of the present disclosure;

FIGS. 8A-8C are schematics showing cells being loaded into an interaction chamber of a device, according to an exemplary embodiment of the present disclosure;

FIGS. 9A-9C are schematics showing cells being loaded into an interaction chamber of a device, according to an exemplary embodiment of the present disclosure;

FIGS. 10A and 10B are brightfield/fluorescent images showing cells being loaded into a device, according to an exemplary embodiment of the present disclosure;

FIGS. 11A-11F depict quantitative characterization of effector:target (E:T) pairing combinations. FIG. 11A is a brightfield view of cancer cells and T-cells in chambers (scale bar: 60 μm). FIGS. 11B-11F are histograms of pairing ratios. FIG. 11B shows results for the entire array. FIG. 11C shows results for an interaction chamber having two particle outlet traps. FIG. 11D shows results for an interaction chamber having three particle outlet traps. FIG. 11E shows results for an interaction chamber having four particle outlet traps. FIG. 11F shows results for an interaction chamber having five particle outlet traps;

FIG. 12 is a flowchart of an example method for loading one or more cells into a device, according to an exemplary embodiment of the present disclosure; and

FIG. 13 is a flowchart of an example method for loading one or more cells into a device, according to an exemplary embodiment of the present disclosure.

### DETAILED DESCRIPTION

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

It should also be noted that, as used in the specification and the appended claims, the singular forms “a,” “an” and “the” include plural references unless the context clearly dictates otherwise. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

It is also to be understood that the mention of one or more method steps does not preclude the presence of additional method steps or intervening method steps between those steps expressly identified. Moreover, although the term “step” may be used herein to connote different aspects of methods employed, the term should not be interpreted as implying any particular order among or between various steps herein disclosed unless and except when the order of individual steps is explicitly required.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter. Additionally, the components described herein may apply to any other component within the disclosure. Merely discussing a feature or component in relation to one embodiment does not preclude the feature or component from being used or associated with another embodiment.

To facilitate an understanding of the principles and features of the disclosure, various illustrative embodiments are explained below. In particular, the presently disclosed subject matter is described in the context of being a microfluidic device for capturing cells. The present disclosure, however, is not so limited and can be applicable in other contexts. For example and not limitation, some embodiments of the present disclosure may improve other fluid delivery systems. Additionally, some embodiments of the present disclosure may improve culture techniques for larger organisms, such as cell aggregates, large multi-cellular organisms, organs, and the like. These embodiments are contemplated within the scope of the present disclosure. Accordingly, when the present disclosure is described in the context of a microfluidic device for capturing cells, it will be understood that other embodiments can take the place of those referred to.

In some embodiments, the present disclosure describes a microfluidic device for capturing cells in one or more interaction chambers. The cells can be captured in a number of ways. In one example, the cells can be added to or included in a fluid flowing in a first direction through a main channel that is in fluid communication with traps on one end of the interaction chamber. Other cells can be added to or included in a fluid flowing in a second direction through the main channel that is in fluid communication with traps at another end of the interaction chamber. As used herein, two elements are in fluid communication with each other, if during normal operation, fluid from one of the elements can pass to the other element. For example, a first interaction chamber can be in fluid communication with a main fluid channel if fluid can pass freely from the main fluid channel to the interaction chamber and vice versa. This is true even if the fluid must also flow through another component that is also in fluid communication with the other components, e.g., a restriction channel and/or a particle trap can fluidly connect the main fluid channel to the interaction chamber so that the main fluid channel and interaction chamber are in fluid communication.

The present disclosure provides devices and methods of use for capturing and pairing different numbers and types of microscale particles. The disclosure provides a microfluidic device that can capture particles in a first set of traps, then can transfer particles to an interaction chamber. This can then be done for a second type of particle using a second set of traps connected to the same interaction chamber. In one embodiment of this disclosure, the device(s) can be applied to studying interactions between different types of human cells. Using the devices and methods, two or more different types of cells can be loaded into the interaction chambers, with defined numbers of each cell type per chamber. This allows for observation of interaction between the cell types.

Various devices and methods are disclosed for capturing and analyzing cells within a microfluidic device, and exemplary embodiments of the devices and methods will now be described with reference to the accompanying figures.

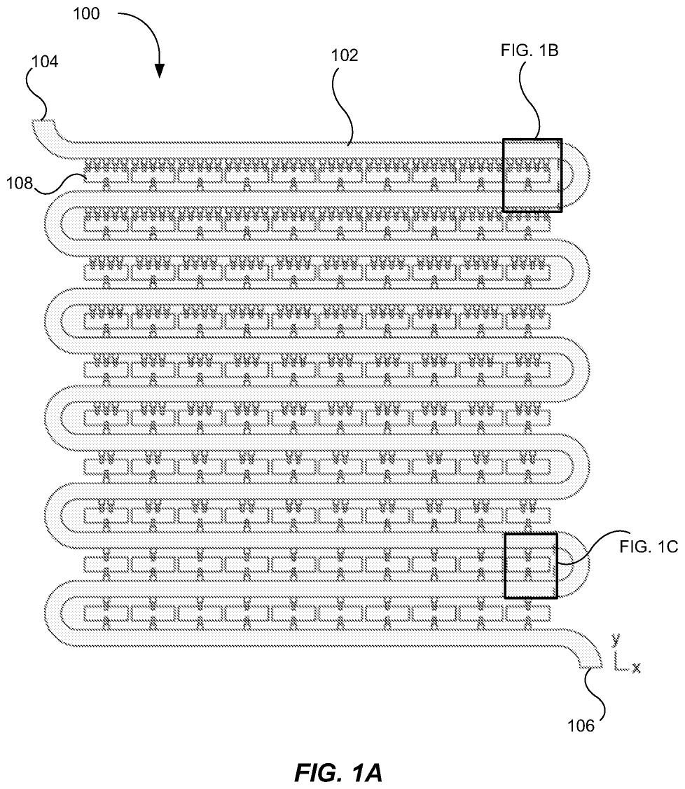

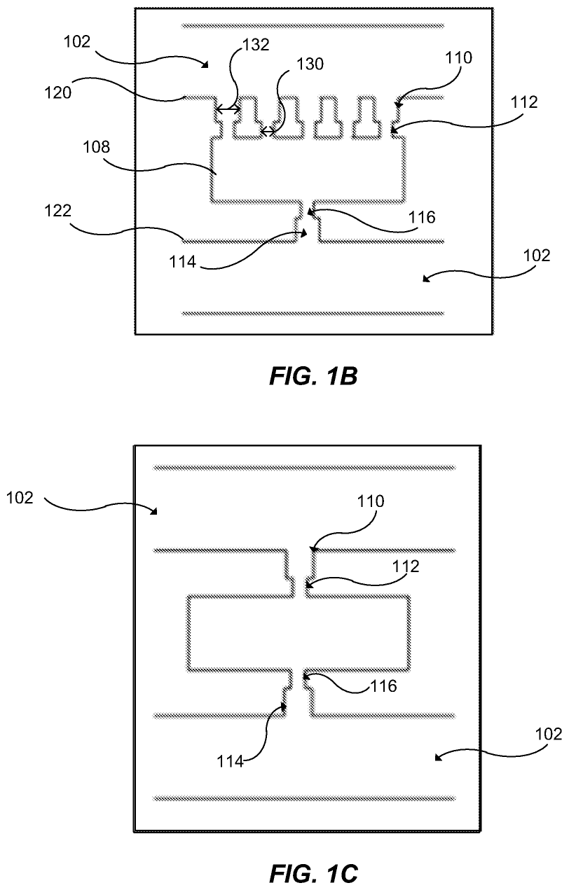

FIG. 1A is a schematic of a device 100 for capturing particles, according to an exemplary embodiment of the present disclosure. FIG. 1B depicts a single interaction chamber 108 of the device shown in FIG. 1A. FIG. 1C depicts another single interaction chamber 108 of the device shown in FIG. 1A. A device 100 may be manufactured in whole or in part from biocompatible materials. Examples of suitable biocompatible materials include, but are not limited to, polydimethylsiloxane (“PDMS”), poly(methyl methacrylate) (“PMMA”), polystyrene, cyclic olefin (co)polymers (“COCs”), silicones, glass, acrylic, polysulfone, and the like or any combination thereof.

The device 100 can include a main fluid channel 102 that has a first opening 104 at a first end of the channel and a second opening 106 at a second end of the channel. As will be described in greater detail below, the first opening 104 can act as a fluid inlet when fluid is flowing through the main fluid channel 102 in a first direction and a fluid outlet when the fluid is reversed to a second direction; the second opening 106 can act as a fluid outlet when fluid is flowing through the main fluid channel 102 in the first direction and act as a fluid inlet when the fluid flows in the second, reverse direction. The main fluid channel 102 can have a serpentine shape, as shown, such that fluid traps can be positioned on opposite sides of the walls of the main fluid channel, as will be described with reference to FIGS. 1B and 1C.

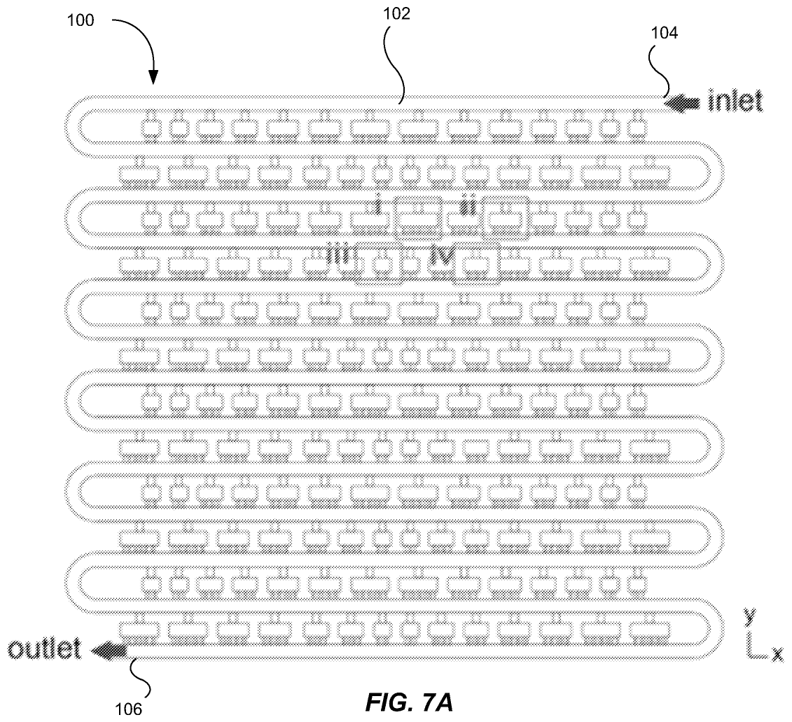

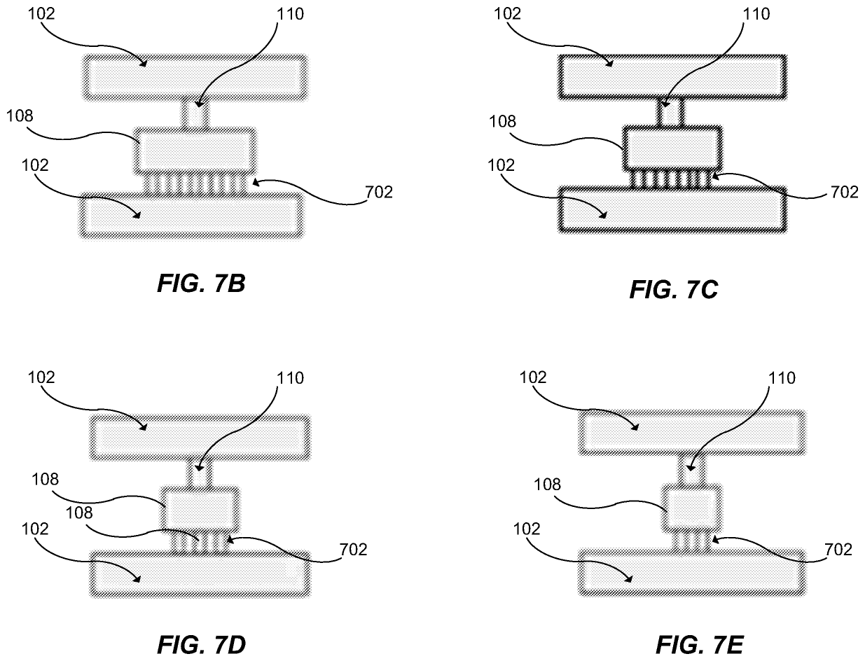

FIG. 1B is a detail example of a trap/chamber embodiment. As can be seen, one or more particle traps can be positioned along a wall of the main fluid channel 102. When reference is made herein to a “trap,” it will be understood to refer to a feature positioned between the interaction chamber 108 and the main fluid channel 102 that can assist in loading a particle into an interaction chamber 108. In some examples, the “traps” can be configured to retain a particle for a period of time until fluid pressure is increased to move the particle from the trap and into the interaction channel, for example through a restriction channel. The example device shown in FIGS. 1A-6C all depict a device having traps that are configured to retain a particle until fluid pressure is increased. In some examples, the “traps” can be configured to pass the cell freely from the main fluid channel 102 into the interaction chamber 108, without an increase in fluid pressure to move the cell through a restriction channel. For example, FIGS. 7A-10B all depict an example wherein a first side of the interaction chamber(s) 108 includes traps leading directly into the interaction chambers, and a second side of the interaction chamber(s) 108 include particle outlet traps (e.g., particle outlet traps 702) that can be blocked by particles within the interaction chamber(s) 108.

Referring again to FIG. 1B, a first particle trap 110 can extend from a first wall 120 of the main fluid channel 102. The first particle trap 110 can be positioned between the main fluid channel 102 and an interaction chamber 108. One or more particle traps can be positioned along another wall of the main fluid channel 102 opposite the first wall 120. For example, a second particle trap 114 can extend from a second wall 122 of the main fluid channel 102. The second particle trap 114 can be positioned between the main fluid channel 102 and the interaction chamber 108. When fluid containing one or more cells is flowing through the main fluid channel 102 in a first direction, a hydrodynamic flow principle enables flow both through the main fluid channel 102 and through the trap/chamber/trap combined channel back to the main fluid channel 102.

In some examples, the device 100 can include restriction channels that have a smaller cross-section than the respective traps. For example, the device 100 can include first restriction channel 112 positioned between the first particle trap 110 and the interaction chamber 108; the first restriction channel 112 can have a first channel cross-section 130 that is smaller than a second channel cross-section 132 of the first particle trap 110. The first restriction channel 112 can enable the first trap 110 to hold a particle until pressure is increased to the fluid to pass the particle through the narrower first restriction channel 112. For example, once a microparticle enters an outer trap (e.g., a first particle trap 110 in this example shown in FIGS. 1A-6C), the microparticle can block additional microparticles from loading into that trap. Once all microparticles of interest have been loaded into the outer traps, additional fluid pressure can be applied to pass the microscale particles into the interaction chamber. The device 100 can also have a second restriction channel 116 positioned between the second particle trap 114 and the interaction chamber 108; the second restriction channel 116 can also have a smaller channel cross-section than the second particle trap 114, as described for the first particle trap and first restriction channel. To load cells from the second particle trap 114 on the other side of the interaction chamber 108, fluid containing one or more cells can be administered from the second opening 106. Once a microparticle enters an outer trap (e.g., a second particle trap 114), the microparticle can block additional microparticles from loading into that trap. Once all microparticles of interest have been loaded into the outer traps in that flow direction, additional fluid pressure can be applied to pass the microscale particles into the interaction chamber 108, where they can interact with cells that were loaded in the first flow direction.

FIG. 1B shows an example trap/chamber embodiment that includes five particle traps 110 on a first side of the interaction chamber 108 and a single particle trap 114 on the other side of the interaction chamber 108, which is in accordance with some embodiments. In this example, five different cells or other particles can be loaded in to the first set of particle traps 110—i.e., when fluid is flowing in a first direction through the main fluid channel 102. Once the five cells are loaded into the interaction chamber 108, fluid can be delivered from the opposite direction and a single cell or other particle can be loaded into the particle trap 114 at the other end of the interaction chamber 108, and then into the interaction chamber 108. This setup can be beneficial in situations wherein one set of particles/cells that are loaded have different sizes than the cell(s)/particle(s) loaded from the other side of the interaction chamber 108. To illustrate using a non-limiting example, in the example trap/chamber embodiment shown in FIG. 1B, five CAR-T cells can be loaded into the first side of the interaction chamber 108 via the first set of particle traps 110. A cancer cell, which can be larger in size than the CAR-T cells, can be loaded to the other end and can be captured by the single, second particle trap 114 on the opposite side of the interaction chamber 108. The five CAR-T cells can then interact with the one cancer cell and can be observed directly within the device 100.

It is contemplated that the first side of the interaction chamber 108 can have any number of first particle traps 110, e.g., one, two, three, four, five, or more particle traps. When the first side of the interaction chamber 108 has more than one trap, each of the particle traps on that side of the interaction chamber 108 can have the same channel cross-section dimensions, as shown. In some examples, the channel cross-section of the more than one particle traps of the first particle traps 110 on the first side of the interaction chamber 108 can vary in size, such that varying sized cells/particles can be loaded into the same side of the interaction chamber 108 via different traps. Further, it is contemplated that the other side of the interaction chamber 108 can have more than one particle trap 114, e.g., one, two, three, four, five, or more particle traps. FIG. 1C shows an example trap/chamber embodiment having only one particle trap 110 at one side of the interaction chamber 108 and one particle trap 114 at the other side of the interaction chamber, which is also in accordance with some embodiments of the present disclosure. In some examples, the channel cross-section of the more than one particle traps of the second particle traps 114 on the second side of the interaction chamber 108 can vary in size, such that varying sized cells/particles can be loaded into the same side of the interaction chamber 108 via different traps. As shown in FIG. 1A, a single device 100 can have numerous interaction chambers 108, and the quantity of particle traps on each of the interaction chambers 108 can differ across the several interaction chambers 108. The example device shown in FIG. 1A includes 100 interaction chambers 108, which is in accordance with some embodiments of the present disclosure.

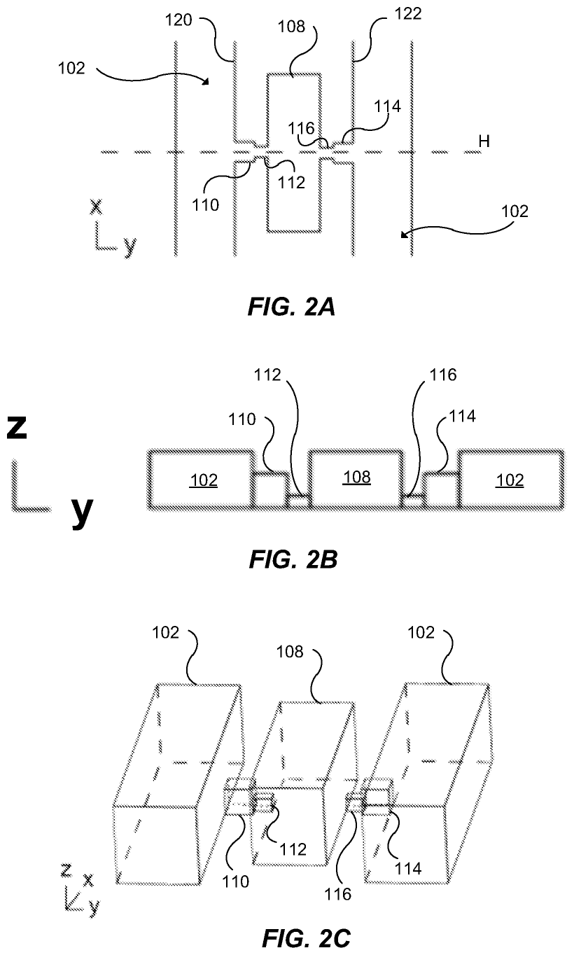

FIGS. 2A-2C are schematics of the layout of an example interaction chamber 108, according to an exemplary embodiment of the present disclosure. FIG. 2A is a top/bottom plan schematic of the interaction chamber 108 and corresponding traps and channels. Reference H shows the direction in which fluid can pass via the trap/restriction channel/interaction chamber. As will be understood, a first particle trap 110 can be “downstream” or “upstream” from a second particle trap 114 with respect to the main fluid channel 102, depending on the flow direction of the fluid.

FIG. 2B is a cross sectional schematic of the interaction chamber 108. The view shows how the main fluid channel 102, first particle trap 110, second particle trap 114, first restriction channel 112, second restriction channel 116, and interaction chamber 108 can be formed into a single substrate. In some examples, at least a portion of the main fluid channel 102, first particle trap 110, second particle trap 114, first restriction channel 112, second restriction channel 116, and interaction chamber 108 can be coplanar, as shown. The coplanarity can be achieved via the manufacturing processes. For example, the device 100 can be molded, etched, and/or manufactured via photolithography into a partially-planar substrate such that the features are made into the substrate, and one surface of the substrate is planar. When the substrate is planar, a slide, such as a glass slide, can be added to the planar surface to perform analysis.

FIG. 2C is a perspective schematic of the interaction chamber 108. The view shows the device flipped from the view shown in FIG. 2B, wherein the planar surface is shown facing downward in the view. The view also shows how the traps (e.g., first particle trap 110 and/or second particle trap 114) can have a larger channel cross-section (e.g., height/width/diameter, depending on the same of the trap) than the restriction channels (e.g., first restriction channel 112 and second restriction channel 116). The example shown in FIG. 2C shows the features to have square and/or rectangular cross-sections. However, the features (e.g., main fluid channel 102, first particle trap 110, second particle trap 114, first restriction channel 112, second restriction channel 116, and/or interaction chamber 108) can have different shapes, e.g., more rounded shapes, depending on the method used to manufacture the device 100. Also, the traps need not have parallel walls, and can also have a funnel-style shape such that the end of the trap closer to the interaction chamber 108 can have a smaller cross section than the end closer to the main fluid channel 102. This design can eliminate the need for a restriction chamber. Further, the dimensions of the features can be modified to accommodate the particles being used in the device. For example, the width, height, etc. of a trap (e.g., second channel cross-section 132 in FIG. 1B) can be from approximately 1 micron to 1.0 mm or larger, depending on the cell being trapped. The width, height, etc. of a restriction channel (e.g., first channel cross-section 130 in FIG. 1B) can be from approximately 1 micron to 1.0 mm or larger, such that the size is smaller than that of the respective trap.

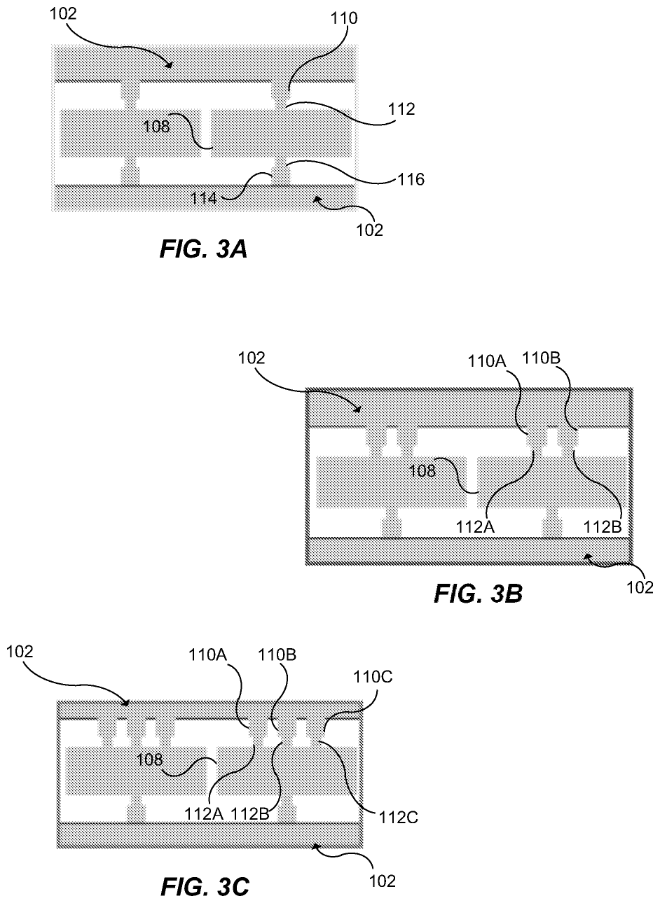

FIGS. 3A-3C are schematics of example interaction chambers 108 of the devices 100 shown herein, according to an exemplary embodiment of the present disclosure. FIG. 3A shows an example device having a single trap on each side of the interaction chamber(s), e.g., a first particle trap 110 on one side and a second particle trap 114 on the other side of the interaction chamber 108. FIG. 3B shows two traps on a first side of the interaction chamber 108. For example, one side of interaction chamber 108 includes a first particle trap 110A and a third particle trap 110B, while the other side of the interaction chamber 108 includes the second particle trap 114. FIG. 3C shows three traps on a first side of the interaction chamber 108. For example, one side of interaction chamber 108 includes a first particle trap 110A, a third particle trap 110B, and a fourth particle trap 110C, while the other side of the interaction chamber 108 includes the second particle trap 114.

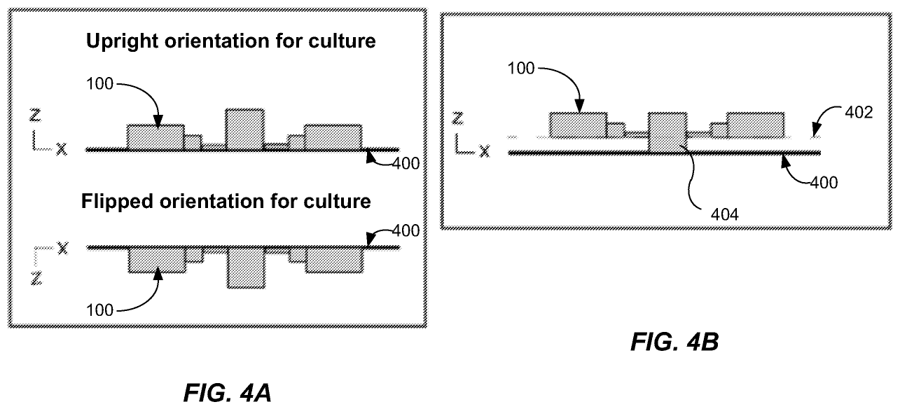

FIGS. 4A and 4B are cross sectional schematics showing example processes for analyzing cells within a device 100, according to an exemplary embodiment of the present disclosure. In FIG. 4A, the method shows how the device can be flipped between loading and analysis. For example, the device 100 can be oriented such that the planar side is upright. A slide 400, such as a glass slide, can be positioned on top of the components of the device along the planar side of the device 100. The slide 400 can provide the uppermost boundary of the channels/chambers such that fluid can flow through each of the components. Once the cells or other particles are captured into the interaction chamber(s) 108, the device 100 can be flipped such that the slide 400 is positioned below the device 100. Cultures or other analysis can be performed directly through the substrate. Alternatively or in addition, culture can be performed through the slide 400.

FIG. 4B is a schematic that shows an alternative example that includes a second layer of substrate 402 that includes an additional interaction chamber section 404 formed into the substrate. The additional interaction chambers section 404 can be formed into the second substrate 402 and molded to or otherwise combined with and/or attached to the first layer of substrate of the device 100. For example, the additional interaction chamber section 404 can include a plurality of chambers that match in size and position with the interaction chambers 108 on the device 100. The second substrate 402 can be closed at one end so as to form the top/bottom surface of the main fluid channel 102, first/second traps, etc. In some examples, the second substrate 402 can only include the additional interaction chamber(s), wherein the additional interaction chamber(s) are open on both ends, one end facing the device 100 and one end being capped by a slide 400.

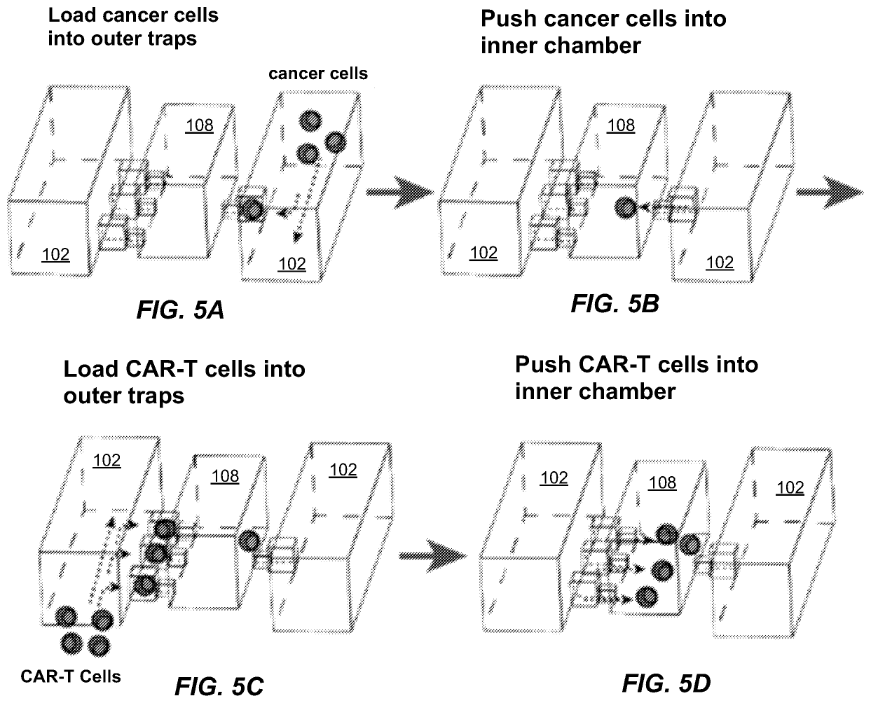

FIGS. 5A-5D are schematics showing an example process for loading cells into an interaction chamber 108 of a device 100, according to an exemplary embodiment of the present disclosure. The schematics shown in FIGS. 5A-5D provide a non-limiting example of a device 100 that includes at least one interaction chamber 108 having three traps/restriction channels at one end and one trap/restriction channel at the other end of the interaction chamber 108. As stated throughout this disclosure, the example interaction chamber 108 shown in FIGS. 5A-5D may be one of a plurality of interaction chambers 108 on a single device 100. In FIG. 5A, cancer cells are added into a main fluid channel 102 via a first fluid flowing in a first direction. A first cell of the cancer cells is trapped into the trap via hydrodynamic flow of the fluid passing not only through the main fluid channel 102 but also through the traps and interaction chamber. In FIG. 5B, the pressure of the first fluid is increased, causing the cancer cell to pass through the restriction channel and into the interaction chamber. The cancer cell is then contained within the interaction chamber 108 for analysis. In FIG. 5C, other cells (e.g., CAR-T cells in this example) are added into the main fluid channel 102 via a second fluid flowing in a second direction. Three CART-T cells are trapped into three respective traps via hydrodynamic flow of the fluid passing not only through the main fluid channel 102 but also through the traps and interaction chamber 108. In FIG. 5D, the pressure of the second fluid is increased, causing the CAR-T cells to pass through the restriction channels and into the interaction chamber. At this point, the three CAR-T cells and the cancer cell can interact, and that interaction can be observed.

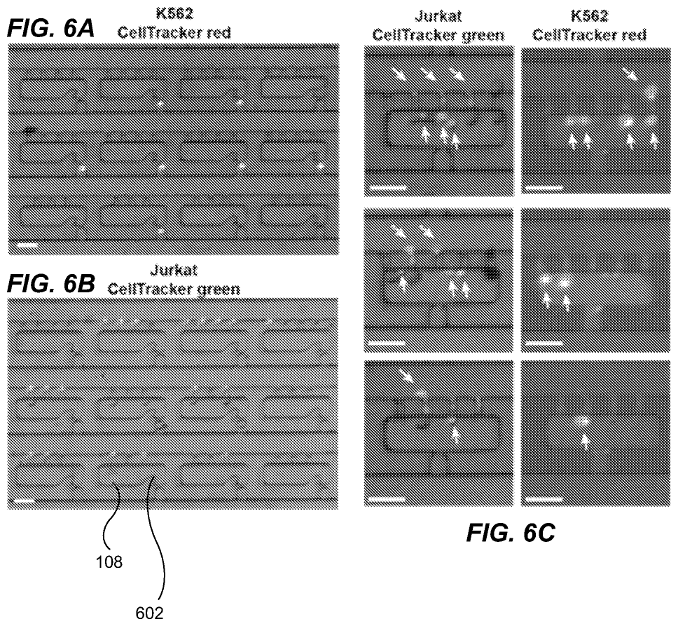

FIGS. 6A-6C are brightfield/fluorescent images showing cells being loaded into a device 100, according to an exemplary embodiment of the present disclosure. FIG. 6A shows K562 cells labeled with CellTracker Red loaded into outer cell traps. In other words, in this example the cells have not yet been loaded into interaction chambers (e.g., the pressure has not been increased to move the cells into the respective interaction chambers). FIG. 6B shows Jurkat cells labeled with CellTracker Green loaded into outer cell traps. In other words, in this example the cells have not yet been loaded into interaction chambers (e.g., the pressure has not been increased to move the cells into the respective interaction chambers). In some examples, each of the one or more interaction chambers 108 can include a chamber ramp 602 positioned proximate one or more of the inlets to the interaction chamber (e.g., proximate the first restriction channel 112, the second restriction channel 116, or both). The chamber ramp 602 can be configured to retain a particle within an interaction chamber 108. Using the image of the example device 100 shown in FIG. 6B, the chamber ramp 602 can be positioned within the interaction chamber 108 proximate the first trap that is used first to capture a cell. Once the cell is captured into the interaction chamber 108, the fluid direction can be reversed to capture cells into the other side of the interaction chamber 108. The chamber ramp 602 can prevent the cell that is already inside the interaction chamber from being inadvertently expelled back through the restriction channel/trap when pressure is increased on the opposite side of the interaction chamber 108.

FIG. 6C shows three example interaction chambers into which both Jurkat and K562 cells have been loaded. Each row is a different interaction chamber. In the left column of images, green fluorescence is overlaid with brightfield and arrows indicate Jurkat cells. In the right column, red fluorescence is overlaid with brightfield and arrows indicate K562 cells. All scale bars are 40 μm.

FIG. 7A is a schematic of a device 100 for capturing particles, according to an exemplary embodiment of the present disclosure. FIG. 7A depicts a device 100 that includes no restriction chambers, meaning the various particle traps around each interaction chamber 108 lead directly from the main fluid channel 102 to the interaction chamber 108. In some implementations, the example device shown in FIG. 7A can be used to capture a plurality of cells suspended in the same fluid. For example, as fluid containing a plurality of cells flows through the main fluid channel 102, the cells will fall into a first particle traps 110 at one side of the interaction chambers 108. Hydrodynamic flow through the first particle traps 110, through the interaction chamber 108, and through the traps/outlets at the other end of the interaction chambers (referred to in this example as one or more particle outlet trap(s) 702) causes the cells to enter the interaction chamber 108 and then block one of the particle outlet traps 702. When the cells block each of the particle outlet traps 702 for a particular interaction chamber 108, the hydrodynamic flow will cease, and no additional cells will enter that particular interaction chamber 108. In this example device, the flow of the fluid need not be reversed, though it can be.

FIG. 7B depicts an interaction chamber of FIG. 7A that includes five particle outlet traps 702, which is in accordance with some embodiments of the present disclosure. In these examples, five (or more) cells or other particles can block the five particle outlet traps 702 before flow ceases into the interaction chamber 108. The interaction of those five cells/particles can then be observed in any manner described herein. FIG. 7C depicts an interaction chamber of FIG. 7A that includes four particle outlet traps. FIG. 7D depicts an interaction chamber of FIG. 7A that includes three particle outlet traps. FIG. 7E depicts an interaction chamber of FIG. 7A that includes two particle outlet traps according to an exemplary embodiment of the present disclosure. It is contemplated that the device can include a plurality of interaction chambers 108, and the interaction chambers 108 can include a varying quantity of particle outlet traps 702.

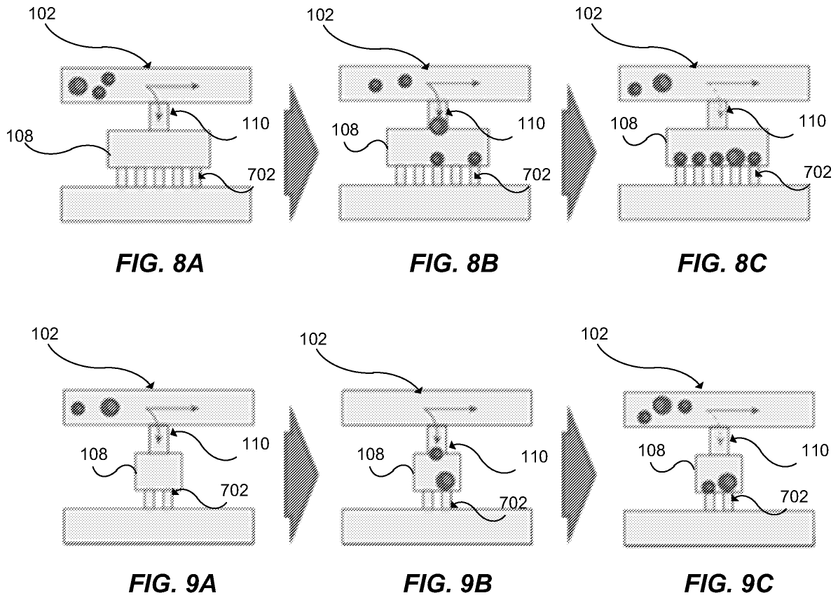

FIGS. 8A-8C are schematics showing cells being loaded into an interaction chamber 108 of a device 100, according to an exemplary embodiment of the present disclosure. The example interaction chamber 108 shown includes particle outlet traps 702. In FIG. 8A, a fluid comprising a plurality of particles (e.g., cells) can be administered into the main fluid channel 102 of the device 100. In FIG. 8B, the particles enter the interaction chamber 108 via the first particle trap 110, and each particle flows to a respective particle outlet trap 702. In FIG. 8C, each of the particle outlet traps 702 are now blocked by a respective particle, and fluid flow can cease to enter that interaction chamber 108. FIGS. 9A-9C are schematics showing cells being loaded into an interaction chamber 108 of a device 100, according to an exemplary embodiment of the present disclosure. In the examples shown in FIGS. 9A-9C, the interaction chamber 108 includes two particle outlet traps 702, which can be blocked by two particles, as shown in FIG. 9C. In FIG. 9A, a fluid comprising a plurality of cells is administered into the main fluid channel 102. In FIG. 9B, the particles enter the interaction chamber 108 via the first particle trap 110, and each particle flows to a respective particle outlet trap 702. In FIG. 9C, each of the particle outlet traps 702 are now blocked by a respective particle, and fluid flow can cease to enter that interaction chamber 108. It is contemplated that a larger quantity of cells may enter an interaction chamber 108 that there are particle outlet traps 702. This may occur if cells enter the interaction chamber 108 but do not fully block one or more of the particle outlet traps 702.

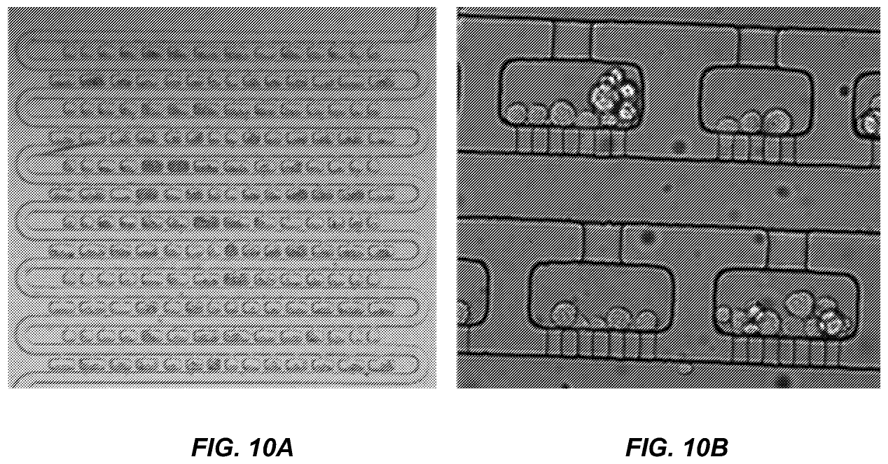

FIGS. 10A and 10B are brightfield/fluorescent images showing cells being loaded into a device, according to an exemplary embodiment of the present disclosure. FIG. 10A provides a zoomed-out view showing fully-loaded array of K562 and Jurkat cells, and FIG. 10B provides a zoomed-in detail, merged.

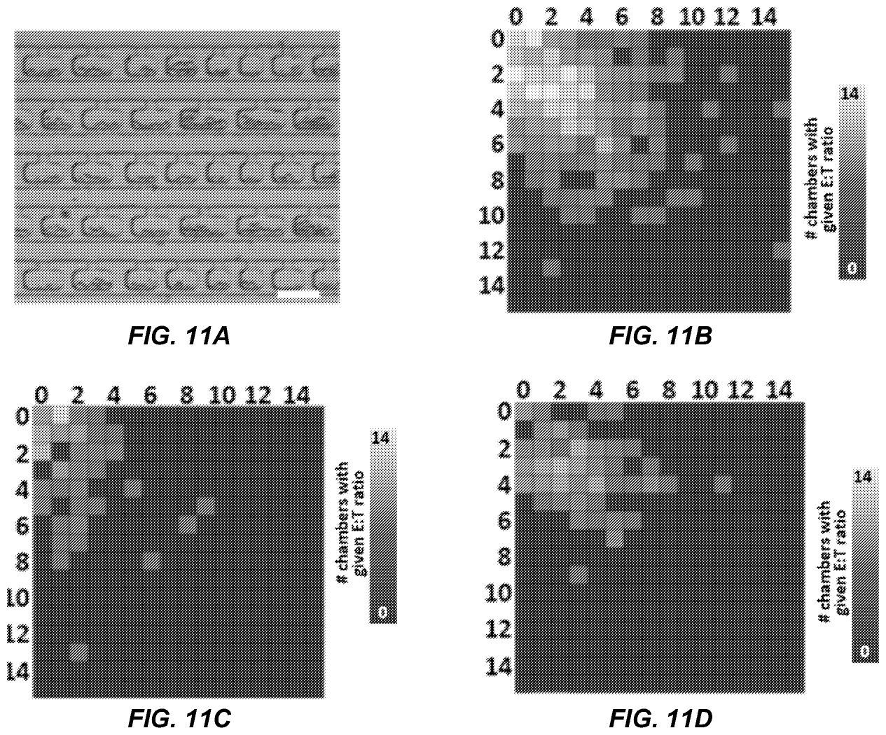

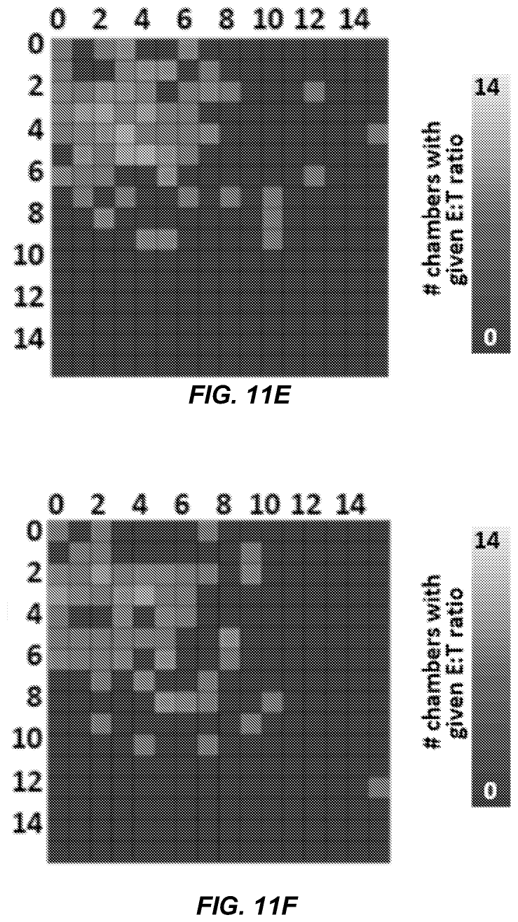

FIGS. 11A-11F depict quantitative characterization of effector:target (E:T) pairing combinations for a device as shown in FIG. 7A. FIG. 11A is a brightfield view of cancer cells and T-cells in chambers (scale bar: 60 μm). FIGS. 11B-11F are histograms of pairing ratios. FIG. 11B shows results for the entire array shown for a device similar to that shown in FIG. 7A. FIG. 11C shows results for an interaction chamber having two particle outlet traps 702. FIG. 11D shows results for an interaction chamber having three particle outlet traps 702. FIG. 11E shows results for an interaction chamber 108 comprising four particle outlet traps 702. FIG. 11F shows results for an interaction chamber having five particle outlet traps 702.

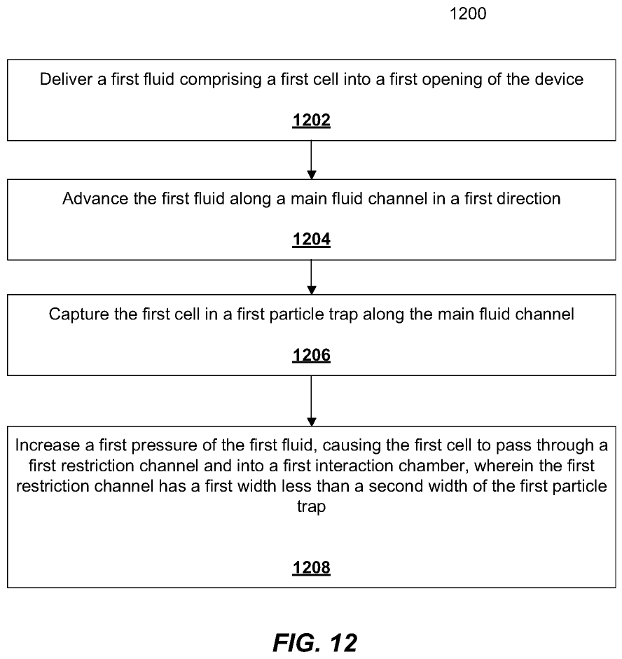

FIG. 12 is a flowchart of an example method 1200 for loading one or more cells into a device, according to an exemplary embodiment of the present disclosure. Method 1200 can include delivering 1202 a first fluid comprising a first cell into a first opening of the device. Method 1200 can include advancing 1204 the first fluid along a main fluid channel in a first direction. Method 1200 can include capturing 1206 the first cell in a first particle trap along the main fluid channel. Method 1200 can include increasing 1208 a first pressure of the first fluid, causing the first cell to pass through a first restriction channel and into a first interaction chamber, wherein the first restriction channel has a smaller cross-sectional dimension than the first particle trap.

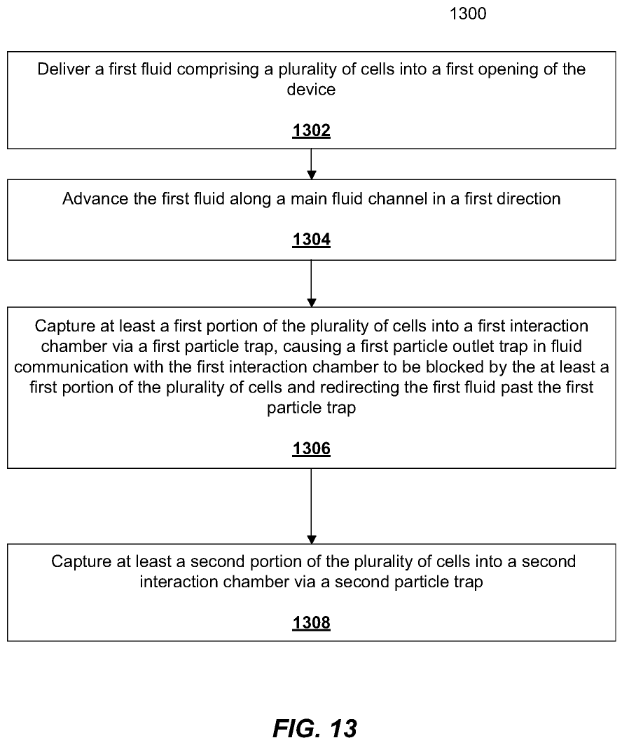

FIG. 13 is a flowchart of an example method 1300 for loading one or more cells into a device, according to an exemplary embodiment of the present disclosure. Method 1300 can include delivering 1302 a first fluid comprising a plurality of cells into a first opening of the device. Method 1300 can include advancing 1304 the first fluid along a main fluid channel in a first direction. Method 1300 can include capturing 1306 at least a first portion of the plurality of cells into a first interaction chamber via a first particle trap, causing a first particle outlet trap in fluid communication with the first interaction chamber to be blocked by the at least a first portion of the plurality of cells and redirecting the first fluid past the first particle trap. Method 1300 can include capturing 1308 at least a second portion of the plurality of cells into a second interaction chamber via a second particle trap.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office, other organizations, and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. Instead, it is intended that the disclosed technology is defined by the claims appended hereto.

1. A device comprising:
a first particle trap extending from a first wall of a main fluid channel and positioned between the main fluid channel and a first interaction chamber;
wherein the first particle trap and a second particle trap are in fluid communication both via the first interaction chamber and via the main fluid channel.

2. The device of claim 1, further comprising:
the main fluid channel;
the first interaction chamber; and
the second particle trap;
wherein the first interaction chamber is in fluid communication with the main fluid channel; and
wherein the second particle trap extends from a second wall of the main fluid channel and is positioned between the main fluid channel and the first interaction chamber.

3. The device of claim 2 further comprising:
a first restriction channel positioned between the first particle trap and the first interaction chamber;
wherein the first restriction channel has a first channel cross-section smaller than a second channel cross-section of the first particle trap.

4. The device of claim 3 further comprising:
a second restriction channel positioned between the second particle trap and the first interaction chamber;
wherein the second restriction channel has a third channel cross-section smaller than a fourth channel cross-section of the second particle trap.

5. The device of claim 4, wherein at least a portion of the main fluid channel, the first interaction chamber, the first particle trap, the second particle trap, the first restriction channel, and the second restriction channel are coplanar.

6. The device of claim 2 further comprising:
a third particle trap extending from the first wall of the main fluid channel and positioned between the main fluid channel and the first interaction chamber.

7. The device of claim 6, wherein the first particle trap and the third particle trap are parallel and coplanar.

8. (canceled)

9. The device of claim 2, wherein the first particle trap is one of a plurality of particle traps extending from the first wall of the main fluid channel and positioned between the main fluid channel and the first interaction chamber; and
wherein the second particle trap is one of a plurality of particle traps extending from the second wall of the main fluid channel and positioned between the main fluid channel and the first interaction chamber.

10.-13. (canceled)

14. The device of claim 2, wherein the first interaction chamber is one of a plurality of interaction chambers; and
wherein the plurality of interaction chambers comprise at least some interaction chambers having different quantities of particle traps.

15. The device of claim 2, wherein the first particle trap is configured to receive a first particle from the main fluid channel and pass the first particle to the first interaction chamber when fluid is flowing through the main fluid channel in a first direction; and
wherein the second particle trap is configured to receive a second particle from the main fluid channel and pass the second particle to the first interaction chamber when fluid is flowing through the main fluid channel in a second direction.

16. The device of claim 2, wherein the first particle trap is configured to receive a first particle from the main fluid channel and pass the first particle to the first interaction chamber when fluid is flowing through the main fluid channel in a first direction; and
wherein the second particle trap is blocked by the first particle when the fluid is flowing in the first direction.

17. The device of claim 2, further comprising a chamber ramp positioned within the first interaction chamber proximate the second particle trap and configured to retain a particle within the first interaction chamber.

18. A method for loading cells into the device of claim 2 comprising:
routing a first fluid comprising a first cell in a first direction through the main fluid channel such that the first cell is captured in the first particle trap and passes to the first interaction chamber; and
routing a second fluid comprising a second cell in a second direction through the main fluid channel such that the second cell is captured in the second particle trap and passes to the first interaction chamber.

19. A method for manufacturing the device of claim 2 comprising molding the main fluid channel, the first particle trap, the second particle trap, and the first interaction chamber into a planar surface of a substrate.

20. A method for manufacturing the device of claim 2 comprising etching the main fluid channel, the first particle trap, the second particle trap, and the first interaction chamber into a planar surface of a substrate.

21. A device comprising:
a main fluid channel having a serpentine shape;
a first interaction chamber in fluid communication with the main fluid channel;
a first particle trap extending from a first wall of the main fluid channel and positioned between the main fluid channel and the first interaction chamber; and
a second particle trap extending from a second wall of the main fluid channel and positioned between the main fluid channel and the first interaction chamber;
wherein the first particle trap and the second particle trap are in fluid communication both via the first interaction chamber and via the main fluid channel.

22. The device of claim 21 further comprising:
a first restriction channel positioned between the first particle trap and the first interaction chamber; and
a second restriction channel positioned between the second particle trap and the first interaction chamber;
wherein the main fluid channel comprises a first opening and a second opening;
wherein the first opening acts both as a first, fluid inlet and a first fluid outlet; and
wherein the second opening acts both as a second fluid inlet and a second fluid outlet.

23. The device of claim 22, wherein one or more of:
the first restriction channel has a first channel cross-section smaller than a second channel cross-section of the first particle trap, and the second restriction channel has a third channel cross-section smaller than a fourth channel cross-section of the second particle trap;
at least a portion of the main fluid channel, the first interaction chamber, the first particle trap, the second particle trap, the first restriction channel, and the second restriction channel are coplanar; and
the first particle trap is configured to receive a first particle from the main fluid channel and pass the first particle to the first interaction chamber through the first restriction channel when fluid is flowing through the main fluid channel in a first direction, and the second particle trap is configured to receive a second particle from the main fluid channel and pass the second particle to the first interaction chamber through the second restriction channel when fluid is flowing through the main fluid channel in a second direction.

24. (canceled)

25. The device of claim 22 further comprising:
a second interaction chamber in fluid communication with the main fluid channel;
a third particle trap extending from the first wall of the main fluid channel and positioned between the main fluid channel and the second interaction chamber, the third particle trap being positioned linearly from the first particle trap along the first wall;
a fourth particle trap extending from the second wall of the main fluid channel and positioned between the main fluid channel and the second interaction chamber, the fourth particle trap being positioned linearly from the second particle trap along the second wall;
a third restriction channel positioned between the third particle trap and the second interaction chamber; and
a fourth restriction channel positioned between the fourth particle trap and the second interaction chamber.

26. (canceled)

27. The device of claim 25 further comprising:
a fifth particle trap extending from the first wall of the main fluid channel and positioned between the main fluid channel and the second interaction chamber, the fifth particle trap being positioned linearly from the first particle trap and the third particle trap along the first wall; and
a fifth restriction channel positioned between the fifth particle trap and the second interaction chamber.

28. The device of claim 22 further comprising:
a third particle trap extending from the first wall of the main fluid channel and positioned between the main fluid channel and the first interaction chamber; and
a third restriction channel positioned between the third particle trap and the first interaction chamber;
wherein a first channel cross-section of the first particle trap is larger than a second channel cross-section of the third particle trap; and
wherein the first particle trap is configured to trap larger cells within a fluid delivered to the first opening than the third particle trap.

29. (canceled)

30. The method of claim 18 further comprising:
increasing a first pressure of the first fluid, causing the first cell to pass through a first restriction channel positioned between the first particle trap and the first interaction chamber and into the first interaction chamber; and
increasing a second pressure of the second fluid, causing the second cell to pass through a second restriction channel positioned between the second particle trap and the first interaction chamber and into the first interaction chamber.

31. The method of claim 30 further comprising:
placing a slide onto a planar surface covering the main fluid channel, the first particle trap, the first restriction channel, the second particle trap, the second restriction channel, and the first interaction chamber;
orienting the device such that the slide is placed below the device; and
performing an analysis of the first cell and second cell through a substrate of the device comprising the first interaction chamber.

32.-33. (canceled)

34. A device comprising:
a main fluid channel having a serpentine shape; and
interaction chambers disposed along a length of the main fluid channel;
wherein a first interaction chamber of the interaction chambers comprises:
a first trap in fluid communication with the main fluid channel at one end and the first interaction chamber at another end; and
first particle outlet traps in fluid communication with the first interaction chamber at one end and the main fluid channel at another end; and
wherein the main fluid channel provides a fluid flow path along the serpentine shape such that the first particle outlet traps are positioned along the serpentine shape of the main fluid channel downstream from the first trap along the fluid flow path.

35. (canceled)

36. The device of claim 34, wherein a second interaction chamber of the interaction chambers comprises:
a second trap in fluid communication with the main fluid channel at one end and the second interaction chamber at another end; and
second particle outlet traps in fluid communication with the second interaction chamber at one end and the main fluid channel at another end.

37. The device of claim 36, wherein the number of second particle outlet traps is fewer than the number of first particle outlet traps.

38. The device of claim 34, wherein the first particle outlet traps are configured to be blocked by cells within the first interaction chamber, thereby stopping flow through the first trap.

39. (canceled)

40. A method for loading cells into a device comprising:
advancing a first fluid comprising a first cell from a first opening of the device and along a main fluid channel in a first direction;
capturing the first cell in a first particle trap along the main fluid channel; and
increasing a first pressure of the first fluid, causing the first cell to pass through a first restriction channel and into a first interaction chamber;
wherein the first restriction channel has a first channel cross-section smaller than a second channel cross-section of the first particle trap.

41. The method of claim 40 further comprising:
advancing a second fluid comprising a second cell from a second opening of the device and along the main fluid channel in a second direction;
capturing the second cell in a second particle trap along the main fluid channel; and
increasing a second pressure of the second fluid, causing the second cell to pass through a second restriction channel and into the first interaction chamber;
wherein the second restriction channel has a third channel cross-section smaller than a fourth channel cross-section of the second particle trap.

42. The method of claim 41 further comprising:
placing a slide onto a planar surface covering the main fluid channel, the first particle trap, the first restriction channel, the second particle trap, the second restriction channel, and the first interaction chamber;
orienting the device such that the slide is placed below the device; and
performing an analysis of the first cell and second cell through a substrate of the device comprising the first interaction chamber.

43.-46. (canceled)

47. A method for loading cells into a device comprising:
advancing a first fluid comprising cells from a first opening of the device and along a main fluid channel in a first direction;
capturing a first portion of the cells in a first interaction chamber via a first particle trap, causing a first particle outlet trap in fluid communication with the first interaction chamber to be blocked by the first portion of the cells and redirecting the first fluid past the first particle trap; and
capturing a second portion of the cells in a second interaction chamber via a second particle trap;
wherein the first portion of the cells further blocks a second particle outlet trap in fluid communication with the first interaction chamber; and
wherein the second interaction chamber has a different quantity of particle outlet traps than the first interaction chamber, thereby requiring a different quantity of cells to block flow into the second interaction chamber.

48.-49. (canceled)
